(19) United States
(12) Patent Application Publication
(10) Pub. No.: US 2022/0047787 A1
(43) Pub. Date: Feb. 17, 2022

(54) Apparatus, System and Methods for Independently Adjusting Double Pump Breast Pump

(71) Applicants: Mother's Milk, Inc., DBA Spectra Baby USA, Davie, FL (US); Min Byung Wook, DBA Uzinmedicare, Seongnam-si (KR)

(72) Inventors: Heidi Humphries, Southwest Ranches, FL (US); Samuel Levey, Coral Springs, FL (US); Min Byung Wook, Seongnam City (KR)

(21) Appl. No.: 17/403,417

(22) Filed: Aug. 16, 2021

Related U.S. Application Data
(60) Provisional application No. 63/065,861, filed on Aug. 14, 2020.

(51) Int. Cl. A61M 1/06

(52) U.S. Cl. CPC A61M 1/064; A61M 1/06; A61M 2205/502; A61M 1/0693; A61M 2210/1007; A61M 2209/088; A61M 2205/50

(57) ABSTRACT

The present disclosure is directed to an improved electric breast pump performance by providing independent adjustment of pumping parameters, such as vacuum and cycle, among others, for each breast when double pumping. The electric breast pump provides the capability to independently select discrete values for each breast so that a separate setting can be set for vacuum and/or cycles or both and can be selected for each breast. These separate settings can be varied and adjusted independently. The improved breast pump can be operated to pump individual breasts, or both breasts simultaneously according to the setting. The improved breast pump utilizes data and information received from previous breast pump sessions to determine and provide improved and optimal settings for future sessions, thereby creating a more efficient breast pump system.

### PRIORITY STATEMENT

This patent application claims the benefit of U.S. Provisional Application 63/065,861, titled APPARATUS, SYSTEM AND METHODS FOR INDEPENDENTLY ADJUSTING DOUBLE PUMP BREAST PUMP, filed Aug. 14, 2020, which application is incorporated entirely by reference herein.

### FIELD OF THE DISCLOSURE

The present disclosure relates to an apparatus, system and methods for an improved breast pump used for expressing and collecting breastmilk. In particular, the present disclosure relates to an electric, double pumping breast pump that will allow for independent adjustment of pumping parameters, including but not limited to vacuum and/or cycles, for each breast separately, to improve the expression and collection of breast milk.

The present disclosure further relates to an apparatus, system and methods for an improved breast pump that utilizes data and information received from previous or particular breast pump sessions to provide improvement, including optimal settings, for future expressing and collecting breast pump sessions, thereby creating a more efficient breast pump system.

### BACKGROUND OF THE DISCLOSURE

Devices for expressing human breast milk are well known. In principle, there are two different types: the first device type is operated manually, i.e. the negative pressure required for the expressing operation is generated by manual actuation of the vacuum pump. The second device type uses electrical power, either through direct connection to utility power or through a battery or another energy source or accumulator.

Electric breast pumps allow milk to be emptied from each breast at the same time in a configuration referred to as “double-pumping”. Double pumping is vastly preferred by mothers because of the reduction in overall time for emptying the breasts of breast milk. In most cases, individual anatomy varies from breast to breast, meaning that one breast will be emptied more efficiently and faster than the other breast. This also means that the optimum or optimal suction settings are not the same from breast to breast.

Current powered breast pump technologies allow for various pumping patterns to improve letdown or stimulation and subsequent milk extraction, these are referred to as “2-phase technology” and are generally standard in most electric breast pumps.

To improve comfort, efficiency, and safety, both the vacuum (suction) levels and the cycles (frequencies) are generally adjustable within selected ranges. A variety of control schema and associated settings are used in setting pumping parameters. These include varying vacuum and cycles according to predetermined curves, fixing cycling at discrete levels and adjusting vacuum, or adjusting both vacuum and cycles independently through a variety of methods. In all cases, the adjustments are common from side to side—meaning each breast is exposed to the same suction levels when double pumping.

As noted previously, each breast does not empty at the same rate, and the lack of independent adjustability leads to extended pumping times with attendant complexity (switching to single pumping at the end of a session) and the general use of suboptimum settings.

Various patents have attempted to address the shortcomings of breast pump systems and various similar devices described herein. One example that pertains to a breast pump with a double breast pump mode is U.S. Pat. No. 8,900,182, issued to Britto, titled Breast Pump And Method Of Use. The '182 patent discloses a breast pump convertible between a single breast pump mode and a double breast pump mode. The breast pump includes a pump and a conduit assembly coupled to the pump and at least one breast shield. The breast pump is operable to convert between the single breast pump mode and the double breast pump mode while maintaining substantially the same vacuum level applied to one of the breasts.

Another example that pertains to a double pump breast pump is U.S. Pat. No. 7,008,400, issued to Silver, et al., titled Diaphragm Pump And Pump For Double-Breast Pumping. The '400 patent discloses a motorized pump that includes a flexible diaphragm fitting within a rigid member, a motor drive mechanism for drawing a puller member attached to the diaphragm away from the rigid member to create a space between the diaphragm and the rigid member and form a negative pressure region within that space, and an outlet communicating with the negative pressure region. In one embodiment, the pump provides a negative pressure in a single chamber which can be used to operate one or two breast shield assembles. In another embodiment, the pump is particularly adapted for double pumping and provides two chambers, which generate vacuum.

Yet another example that pertains to a double pump breast pump is Spanish Patent No. 2,614,933, issued to Luzbetak, et al., titled Connector For Use In Single And Double Milk Extraction. The '933 patent discloses a “connector (200), especially for use with a single or double milk extraction system, including a manifold (202), said manifold including a three-way passage formed therethrough, said manifold (202) a first orifice (204), a second hole (206), a third hole (208) and a fourth hole (210), said first, second and third holes (204, 206, 208) being in fluid communication with said three-way passage, and the fourth orifice (210) not being in fluid communication with said three-way passage; a first tube (212) connected to and in fluid communication with said first hole (204), a second tube (216) connected to and in fluid communication with said second hole (206); and a third tube (220) connected to and in fluid communication with said third hole (208), wherein said third tube (220) is sized and shaped to be able to connect to a vacuum source, characterized in that said first tube (212) ends with a first plug element (214), said second tube (216) ends with a second plug element (218), said fourth hole (210) is sized and shaped to receive one of said first plug element (214) and said second plug element (218) to allow a predetermined amount of fluid to pass through an escape path (A, B) when received, where said escape path (A, B) is formed in said manifold (202).

None of these references disclose, suggest or teach an improved breast pump system that address the disadvantages and limitations of current breast pumps and breast pump systems, which the present disclosure encompasses. At a minimum, none of these references disclose a pump or system that allow for independent adjustment of pumping parameters, including but not limited to vacuum and cycles, for each breast independently. Additionally, none of the present pumps or systems utilize data and information from a particular breast pumping or expressing session to improve future expression sessions, thereby creating a more efficient and optimal breast pump and system. As such, none of these references successfully addresses the shortcomings addressed by the present disclosure.

Accordingly, what is needed is an improved breast pump apparatus, system and related methods that allow for independent adjustment of pumping parameters, including vacuum and cycles, for each breast separately, and that utilize data and information from previous or particular breast pump session or sessions to improve future breast pumping sessions. The present disclosure satisfies these needs.

### SUMMARY OF THE DISCLOSURE

In general, and in order to solve the above-mentioned shortcomings in the field of breast pump technology, the present disclosure relates to improvements in electric breast pump performance by providing for independent adjustment of pumping parameters for each breast when double pumping. Additionally, the present disclosure monitors data and information from a particular breast pump session or sessions to improve future sessions.

In particular, the present disclosure relates to an apparatus, system and methods for improved electric breast pump performance by providing independent adjustment of pumping parameters, such as vacuum and cycle, among others, for each breast when double pumping. In doing so, an electric breast pump and system is disclosed with the capability to independently select discrete values for each breast, manually or automatically, so that a separate setting can be set for vacuum and/or cycles or both. These separate settings can be varied and adjusted independently, or synchronized such that the settings are tied to each other and are adjusted in parallel. The improved breast pump can be operated to pump individual breasts, or both breasts simultaneously according to the setting.

The present disclosure further relates to an apparatus, system and methods for improved electric breast pump performance by providing (either manual or automatic) independent adjustment of pumping parameters for each breast when double pumping, and in which timers can be incorporated to control the length of pumping sessions. These timers may be set at the same or different values for each breast. These timers may be individually set by the user or derived from vacuum level data from the prior pumping session. As such, the settings for prior pumping sessions may be stored locally in the device, or recorded and/or remembered into a database, and recalled as necessary for subsequent sessions. These settings may be individually set by the user manually or derived from the prior pumping session automatically. Records may be kept for individual levels for each breast or aggregated information covering the pumping session at the individual level. The data may be collected for individual pumping sessions or over multiple sessions. In doing so, the information obtained can be incorporated into the next pumping session and used for future sessions. New or updated information can be obtained during subsequent pumping sessions to assist in determining the optimal settings or parameters for an individual's pumping session.

Additionally, the present disclosure relates to improved breast pump performance by providing independent adjustment of pumping parameters for each breast when double pumping, utilizing multiple pre-programmed curves individually selectable for each breast so that a preprogrammed curve, utilizing dynamic vacuum and/or cycles, among other controls, can be selected for each breast. These preprogrammed curves can be varied and adjusted independently, and the device can be operated to pump one, or both breasts simultaneously according to the settings entered.

The present disclosure further relates to an improved breast pump that provides a more efficient and speedier session by providing independent adjustment of the suction and frequency during the letdown and stimulation phases of each session, along with control and adjustment at the start, during the middle and/or at the end of the session to obtain a more optimal and efficient session.

The present disclosure further relates to an apparatus, system and methods for improved electric breast pump performance by providing independent adjustment of pumping parameters for each breast and utilizing one or more pump aggregates using either mechanical or electrical controllers designed in such a way as to allow independent adjustment of suction and/or cycles for each breast during letdown or stimulation phases of each session. These controls are configured with the ability to make independent adjustments at the start, middle, and/or end of a pumping session.

The present disclosure further relates to an electric breast pump that provides a constant source of vacuum at a base cycling rate to an individual controller which bleeds off or adjusts the vacuum level and/or cycling rate at one or both breasts to allow the pumping patterns at one or both breasts to be individually varied according to the individual's needs. The controller can be mechanical and/or electronic and can be located at or in the breast pump, at the breast shield assembly, or anywhere in between.

The present disclosure further relates to an apparatus, system and methods for improved electric breast pump performance by providing independent adjustment of pumping parameters for each breast and utilizing a user interface to provide the controls for the embodiments disclosed herein. The user interface can be mechanical or electronic, or a combination of both, and can be located at or in the breast pump, at the breast shield assembly, anywhere in between (in the supply line by incorporation into the vacuum or supply tubing), or even be accessible remotely through an app on a phone, personal computer, or other locations.

As such, and merely as an example, the user interface may be located internal to the pump assembly, with remote control capability through an external device, either one made specifically for the application or with an app or control program loaded onto appropriate hardware, including a mobile telephone.

Additionally, the user interface may be located external to the pump assembly, with the controls located on the pump assembly itself, and the user interface may be located external to the pump assembly, with controls on the user interface itself. Any combination of locations for the user interface and the controller can be incorporated. The user interface allows for adjustments and monitoring of the settings used.

These and other aspects, features, and advantages of the present disclosure will become more readily apparent from the attached drawings and the detailed description of the preferred embodiments, which follow.

### DRAWINGS

The preferred embodiments of the disclosure will be described in conjunction with the appended drawings provided to illustrate and not to the limit the disclosure, where like designations denote like elements, and in which:

FIG. 1 shows a standard breast pump in accordance with the prior art;

FIG. 2 shows standard tubing in accordance with the prior art;

FIG. 3 shows standard backflow protectors in accordance with the prior art;

FIG. 4 shows standard flanges in accordance with the prior art;

FIG. 5 shows standard duckbill valves in accordance with the prior art;

FIG. 6 shows standard wide neck bottles in accordance with the prior art;

FIG. 7 shows a standard 12-volt AC adapter in accordance with the prior art;

FIG. 8 shows an improved breast pump apparatus, system and method in accordance with one embodiment of the present disclosure;

FIG. 9 shows the user interface of an improved breast pump apparatus, system and method in accordance with one embodiment of the present disclosure;

FIG. 10 shows the user interface of an improved breast pump apparatus, system and method in accordance with one embodiment of the present disclosure;

FIG. 11 shows a functional block diagram for an improved breast pump apparatus, system and method in accordance with one embodiment of the present disclosure;

FIG. 12 shows a flow chart for an improved breast pump apparatus, system and method in accordance with one embodiment of the present disclosure.

### DETAILED DESCRIPTION

Referring to the drawings, wherein like reference numerals refer to the same or similar features in the various views, the present disclosure pertains to an improved breast pump apparatus, system and related methods in which, in the preferred embodiment, an electric breast pump improves overall pumping performance by providing independent adjustment of pumping parameters, such as vacuum and cycle, among others, for each breast when double pumping.

To achieve these improvements, the electric breast pump provides the capability to independently select discrete values for pumping each breast separately so that a distinct calibration can be set for vacuum or cycle or both for each breast. These distinct (or in some cases, the same) settings can be varied and adjusted independently to improve and optimize the overall pumping session. The improved breast pump can be operated to pump individual breasts, or both breasts simultaneously according to the setting. Additionally, the settings on the improved breast pump can be synchronized, when desired, so that the both breasts are pumped with the same vacuum and cycle.

The present disclosure comprises numerous components along with a user interface to achieve the desired functionality described herein, which also includes the software program to control the hardware and run the system.

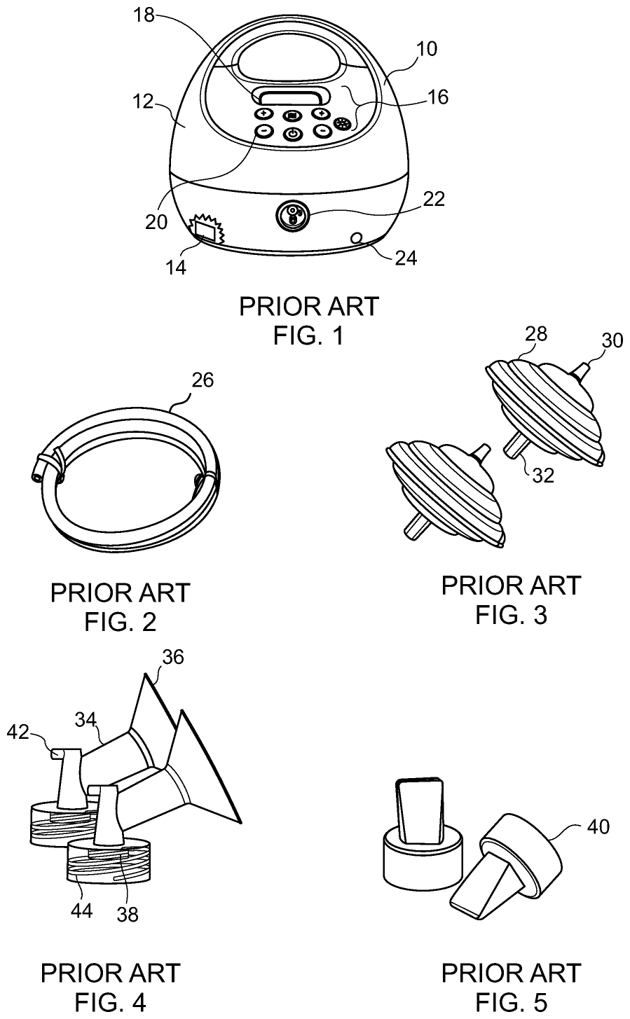

FIG. 1 shows an exemplary standard breast pump 10, in this case, the exemplary standard breast pump 10 is the Spectra S1 Plus Breast Pump distributed by Mother's Milk, Inc. dba Spectra Baby USA (see https://www.spectrababyusa.com/store/s1plus-/). The exemplary Spectra S1 Plus breast pump 10 is portable and rechargeable, and includes a massage mode that simulates the natural suckling of baby. The exemplary Spectra S1 Plus breast pump 10 along with other accessories as described herein can provide backflow protection to keep breast milk from harmful bacteria, and adjustable suction and cycling levels to provide a more natural and comfortable flow of milk.

The standard breast pump 10 comprises a housing 12, a pump 14, a user interface 16 that comprises a display 18, and a plurality of switches 20. The standard breast pump 10 further comprises a tubing connection port 22, and a power connection 24, as understood by one having ordinary skill in the art. In use, the breast pump 10 is connected to the breast in order to provide breast pumping functionality, i.e., pumping out or expressing milk from the breast for later consumption by the baby.

To provide the breast pumping functionality, a standard breast pump 10 may include a number of components making up a breast pump system. FIGS. 2 through 7 show some of the components that make up the breast pump system.

FIG. 2 shows the tubing 26 that the breast pump 10 utilizes to transfer the suction created by the pump 14 to the breast (not shown) for expressing the milk. Tubing 26 can come in various lengths and diameters, depending on user needs or engineering requirements, and to provide appropriate access to the end user. The tubing 26 is attached to the tubing connection port 22 on the breast pump 10, which connects internally to the pump 14.

FIG. 3 shows backflow protectors 28 that are used to prevent milk from entering the tubing 26, thereby minimizing or negating the chance of bacteria forming in the tubing 26. Each backflow protector 28 contains a backflow connector 30 for attachment to the tubing 26, and a backflow attachment device 32 for connecting to a breast flange 34. The backflow protector 28 also includes a flexible membrane (not shown), which is critical to the protection function as understood by one having ordinary skill in the art.

FIG. 4 shows breast flanges 34 having a breast cup 36 for connection to the breast for expressing milk from the breast. Each breast flange 34 has a valve connector 38 for connecting to a duckbill (or similar) valve 40 (shown in FIG. 5) and a flange connector 42 for connecting the breast flange 34 to the backflow attachment device 32 on the backflow protector 28. Additionally, each breast flange 34 has a female screw connector 44 for attachment to a bottle 46 and a valve connector 38.

FIG. 5 shows the duckbill valve 40 that is connected to the breast flange 34 at the valve connector 38 on the breast flange 34. The duckbill valve 40 can be attached to the breast flange 34 either by the threaded valve connector 38 or by using a press fit method as understood by one having ordinary skill in the art. FIG. 6 shows the wide neck bottles 46 containing a male screw connector 48 to allow the wide neck bottles 46 to be screwed into the female screw connector 44, which will locate the duckbill valve 40 inside the wide neck bottle 46 when assembled for pumping.

FIG. 7 shows the 12-volt AC adapter 50 that provides power to the breast pump when the plug side 52 of the 12-volt AC adapter 50 is plugged into a standard outlet and the power side 54 is plugged into the power connection 24 on the breast pump 10.

In use, the components are attached to each other and to the breast pump 10, as described below, so that the breast pump 10 is powered by the 12-volt AC adapter 50 connected at the power connection 24. The tubing 26 is connected at one end to the tubing connection port 22 on the breast pump 10, and at the other end to the backflow protector 28 at the backflow connector 30. The back flow attachment device 32 on the backflow protector 28 is attached to the breast flange 34 at the flange connector 42. The duckbill valve 40 is attached to the breast flange 34 at the valve connector 38 and the wide neck bottle 46 is attached over the duckbill valve 40 using the male 48 and female 44 screw connectors.

With the aforementioned setup, when the breast pump 10 is turned on and set using the switches 20 on the user interface 16, the pump 14 of the breast pump 10 will create a vacuum thereby creating suction through the tubing 26 to the breast cup 36. When the breast cup 36 is placed onto the breast, the cyclic suction will allow the breast to express milk from the breast through the duckbill valve 40 and into the wide neck bottle 46. The backflow protector 28 will prevent milk from escaping through and into the tubing 26.

Additionally, the breast pump 10 may be configured to allow for double pumping. In a double pump breast pump 10, both breasts are allowed to express milk at the same time using a single pump. As described herein, in double pump breast pumps 10, the suction provided at each breast is the same disregarding that each breast may express at different rates. Using the same suction for both breasts decreases optimal overall expressing during a session.

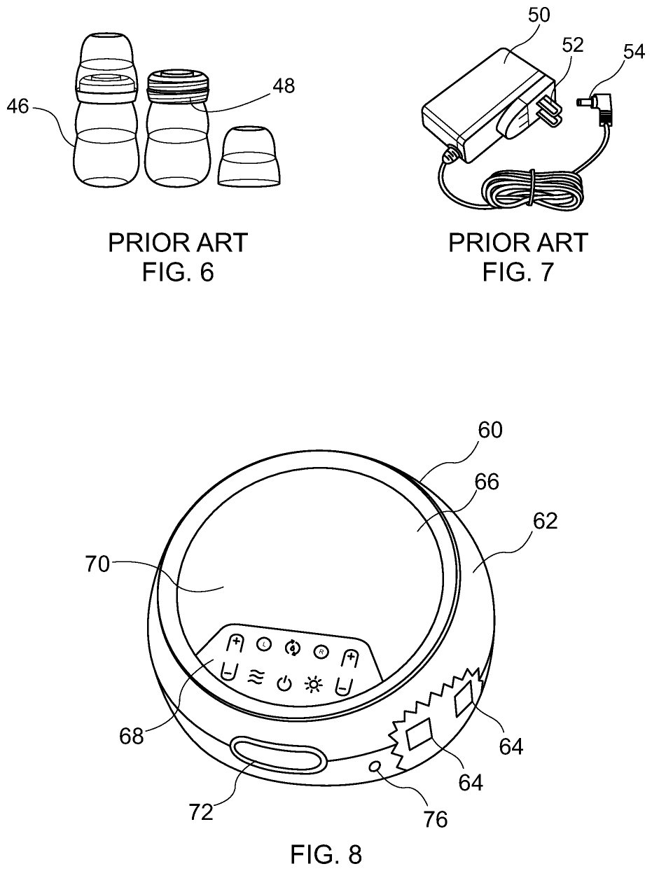

FIG. 8 shows a novel breast pump 60 in accordance with the present disclosure, which is an improved electric breast pump 60 that optimizes overall pumping performance by providing independent adjustment of various pumping parameters and characteristics, for example, vacuum and/or cycle parameters, for each breast when double pumping. The novel breast pump 60 in accordance with the present disclosure also is configured to monitor and/or control breast pump sessions in order to provide a similar experience for each breast pump session, or in order to determine the optimal parameters for each pump sessions, including incorporating different parameters for previous or optimal settings for each breast. Previous settings or parameters may include sound mode, phase mode, massage or expression mode, and/or cycle speed or rate, as described herein. These settings can be used or changed depending on the user's desires. An exemplary description of the particular settings and control of those settings can be seen in the Spectra Synergy Gold Dual Powered Electric Breast Pump instruction manual located at https://www.sepctrababyusa.com/wp-content/uploads/2021/01/SG-Synergy-Gold-Manual.pdf, and incorporated by reference herein.

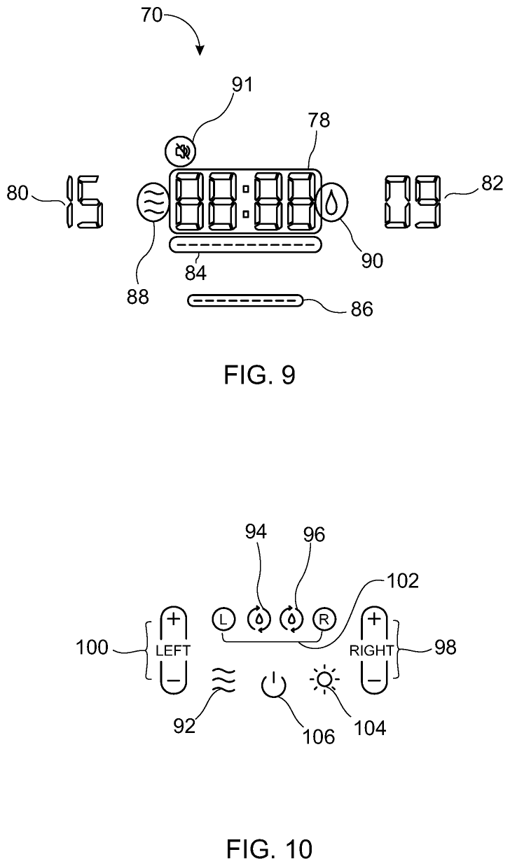

To achieve the improvements disclosed herein, the electric breast pump 60 comprises a housing 62, which houses a printed circuit board comprising a microprocessor, a memory and other components (not shown) as understood by those having ordinary skill in the art. The microprocessor and memory comprise software used to control the breast pump 60 and in particular, the microprocessor and memory are coupled to the user interface (FIGS. 9 and 10), and configured to display the breast pump session parameters, and to allow the user to set parameters, using the user interface. The microprocessor and memory also allow for previous pump session parameters to be displayed and set for the next breast pump session and for determining the optimal breast pump session parameters for each breast and for each user, for example by monitoring and recording the vacuum and cycle settings, and using the timer, to determine the optimal settings, once the breast pump session has been completed.

The housing further protects one or more pumps 64, and which houses and provides the user with a user interface 66, which allows the capability to independently select settings or switches 68 for effectuating the pumping of each breast separately. In the preferred embodiment, two motors or pumps 64 are utilized, controlled by the user interface 66, and in which the vacuum is adjusted independently from side to side. In this embodiment, the cycle settings remain the same. In an alternative embodiment, as described herein, both the vacuum and cycle can be independently adjusted using one or more pumps 64.

The user interface 66 of the breast pump 60 also comprises a display 70, which provides the user with important information about the current pumping session, and also previous pumping sessions, to the extent necessary. Information pertaining to the current or previous pumping sessions can be obtained in numerous ways. The amount of breast milk expressed can be compared to the time to express the breast milk along with the settings for that session. The amount of breast milk expressed for a particular session can be obtained automatically or through user input of the amount. Over time, the optimal settings can be calculated based on multiple (previous) sessions. The results can be displayed in graphical or numerical formats, or by using an optimization curve. Using these results, the user or the system can determine the optimal settings for the current breast pumping session. Additionally, the time of day or night, along with other external factors, can be utilized to further improve the optimal results.

The independently selected settings allow for a separate setting during a session for vacuum or for cycle or for both for each breast. These settings can be varied and adjusted independently to improve and optimize the overall pumping session and to monitor and record the settings for improved optimization (or continued optimal settings) for each session or for future sessions. The improved breast pump can be operated to pump individual breasts, or both breasts simultaneously according to the setting. Additionally, the different settings can be synchronized in order to allow for a single adjustment to control both sides in parallel, as described herein.

The breast pump 60 also comprises a tubing connector port 72 for connecting one or more tubing 26 for transferring the vacuum and suction from the pump 60 to the breast. The breast pump 60 also comprises a power connection 76 for connecting power to the breast pump 60. The breast pump 60 can also include an internal battery (not shown) for charging so that the breast pump does not have to be tethered to a wall outlet when used, providing, for example, a three-hour battery life. Additionally, the breast pump 60 can be configured for inductive or cordless charging (not shown), as understood by one having ordinary skill in the art.

FIG. 9 shows the display 70 in accordance with the preferred embodiment of the present disclosure. The display 70 provides the user with important information about the session, including the running time 78 for the session, a first vacuum level 80, a second vacuum level 82, a first cycle level 84, a second cycle level 86, a massage mode indicator 88, an expression mode indicator 90, a mute indicator 91, among others.

FIG. 10 shows the switches or settings 68 in accordance with the preferred embodiment of the present disclosure. The switches 68 provide the user with the control for the session, including buttons or selectors for the massage/expression mode 92, first adjust cycle rate 94, second adjust cycle rate 96, first adjust vacuum level 98, second adjust vacuum level 100, left/right pump switch 102, night light on/off switch 104 and power on/off 106. As an example, the cycle rate can be set for cycles per minute or similar.

The massage/expression mode, as understood by one having ordinary skill in the art, is usually used at the beginning of the pumping session or whenever there is a need to generate a letdown during a pumping session. The expression mode is used after the letdown begins.

The cycle rate or cycle speed is measured in cycles per minute (CPM) on the breast and refers to how many times the pump suckles and releases per minute. Changing the cycle speed can make a dramatic difference to successful expressing for some women.

As detailed herein, the preferred embodiment does not allow for separate adjustments for the cycle setting for each breast. Instead, the same cycle setting will apply to both breasts. In an alternative embodiment, both the vacuum and cycle settings can be adjusted separately for each breast. The vacuum level or vacuum suction setting level refers to how strongly the pump will suckle; a higher number refers to a higher vacuum.

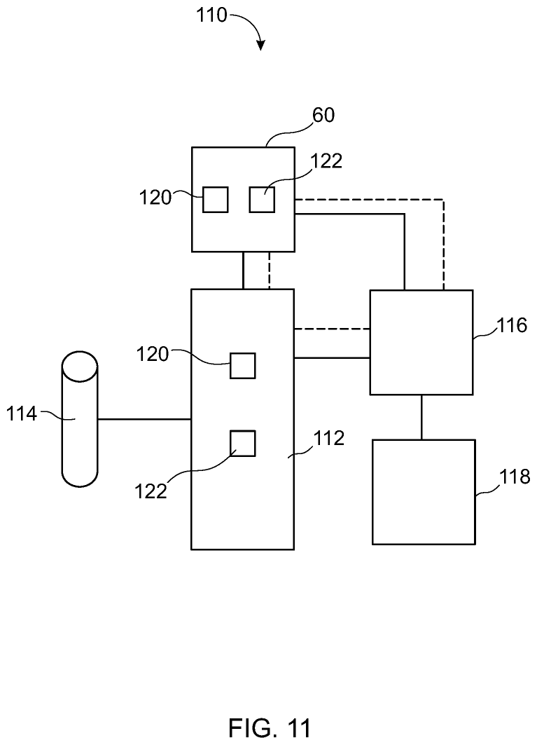

As a non-limiting example, FIG. 11 shows a functional block diagram view of an exemplary breast pump system 110 comprising an electric breast pump 60 for improving performance by providing independent adjustment of pumping parameters for each breast, such as vacuum and cycle, among other parameters when double pumping. The electric breast pump 60 has the capability to independently select discrete values for each breast so that a separate setting 68 can be set for vacuum and/or cycles or both, and can be selected for each breast. Separate settings 68 can be varied and adjusted independently so that the breast pump 60 can be operated to pump individual breasts, or both breasts simultaneously according to the setting.

Each breast pump 60 of the breast pump system 110 utilizes a breast pump server 112, a breast pump database 114, a breast pump application programming interface (“API”) 116, and a system user access 118. The system user access 118 is where users and programmers can access the breast pump system 100 for monitoring and upgrading the software, as necessary, among other functions. The system user access 118 of the breast pump system 110 can be accomplished at the location of the breast pump 60 or remotely depending on the user's needs.

The present disclosure will be described with reference to embodiments in which the breast pump system 110 uses information from one or more pumping sessions to optimize future pumping sessions. The user's access to the breast pump system 110 is through connection to the breast pump API 116. It should be understood, however, that the present disclosure is not limited to the preferred embodiment detailed herein; rather, the system, methods and functionality illustrated and described herein may be incorporated in other ways and still fall under the scope of the present invention.

As an example of the above, a user may use one application program (“app”) on a smart phone to access information about the breast pump 60 and to manually control the breast pump 60, and a separate programmer may use an app to upgrade the system software. Accordingly, the users of the breast pump system 110 may access the breast pump API 116 through the breast pump server 112 or through the system user access 110.

The breast pump system 110 (which may be referred to herein simply as “the system 110”) may include and provide a graphical user interface (GUI) having a number of features described herein. Portions, or all, of the GUI may be provided by the breast pump server 112, in an embodiment. Accordingly, in an embodiment, the breast pump server 112 may be configured to perform one or more operations, methods, etc., described herein that enable various control, calculations and determinations for the system 110.

The breast pump server 112 may be configured to perform a number of functions to assist breast pump system 110 users in their decisions. For example, the breast pump server 112 may be configured to provide optimal pumping session parameters, based on previous session data. The routines, programs and protocols may be obtained from the breast pump server 112, in an embodiment, from the breast pump API 116 and/or from the user access 118. The breast pump system 110 can be used to configure the breast pump 60 either automatically or programmatically as described herein, or the user can configure the breast pump 60 manually as needed through the user access 118 and the API 116.

The breast pump server 112 may be further configured to store information or data and to retrieve that information or data from the breast pump database 114. Data stored in the breast pump database 114 may include previous settings and session information for one or both breasts, including previous breast pump 60 configurations based on that data.

The breast pump database 114 may be or may include one or more data repositories including, but not limited to, one or more databases and database types as well as data storage that may not necessarily be colloquially referred to as a “database.” The breast pump database 114 may be configured to store the information or data described herein, and programs that may be performed through the breast pump system 110, along with similar information related to the needs of the breast pump system 110.

The breast pump server 112 may be in electronic communication with the breast pump system 110 to obtain and deliver updated information, programs and routines, and other information, in an embodiment. Further, the breast pump server 112 may be a single server or multiple servers acting in a redundant or additive capacity, and may be located in remotely from or in close proximity to the breast pump system 110 or the particular breast pump 60.

The breast pump server 112 and the breast pump 60 will include separate processors 120 and memories 122. The processor 120 may be any appropriate processing device, and the memory 122 may be any volatile or non-volatile computer-readable memory. The memory 122 may be configured to store instructions that embody one or more steps, methods, processes, and functions of the breast pump server 120 described herein. The processor 120 may be configured to execute those instructions to perform one or more of the same steps, methods, processes, and functions. Additionally, the processor 120 may be coupled to the pump 64 for providing optimal breast pumping settings. The breast pump server 112 may be or may include a personal computer or mobile device (e.g., tablet, smartphone), in an embodiment.

Instead of, or in addition to, a processor 120 and memory 122, the breast pump server 112 and the breast pump 60 may include a programmable logic device (PLD), application-specific integrated circuit (ASIC), or other suitable processing device (not shown).

The programs and information described herein may be provided, in an embodiment, by the breast pump server 112 (e.g., on a software-as-a-service (SaaS) basis). Storage and retrieval of data displayed in the breast pump system 110, and calculations performed by or under the system 110 may be performed by the breast pump server 112.

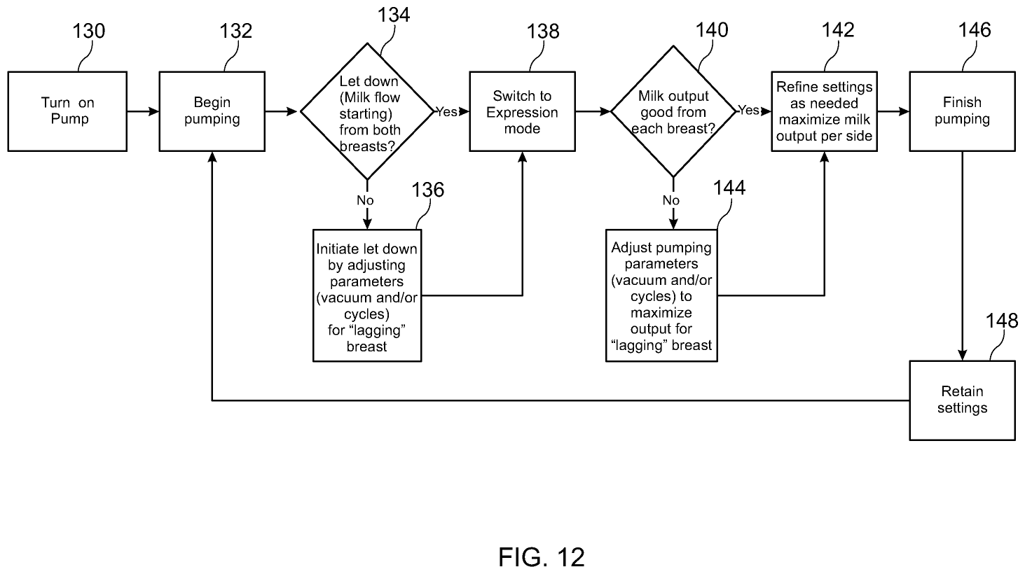

FIG. 12 shows a flow chart incorporating the method of the improved electric breast pump 60 that optimizes overall pumping performance by providing independent adjustment of vacuum and/or cycle parameters, for example, for each breast when double pumping. After the breast pump 60 has been configured as described above, and has been connected to power through the power connection 76, the correct size breast cups are placed on each breast. At step 130, the user turns on the breast pump 60, although the breast pump 60 can be turned on before the breast cups are placed on to the breasts. At step 132, the pump 64 on the breast pump 60 begins pumping, creating a suction through the tubing 26, to the breast flange 34 and to the breast as described herein.

At step 134, determine if letdown (milk flowing) from both breasts. If so, move to step 138. If not, move to step 136. At step 136, the user adjusts the parameters (vacuum strength 98, 100 and/or cycles per minute 94, 96) to initiate letdown in the lagging breast. Once initiated, move to step 138. The system 110 can monitor and record the various parameters, including vacuum strength, cycle and time to finish, among other parameters, for determining the optimal settings.

At step 138, the user switches the massage/expression switch 92 to expression mode. Next, at step 140, the milk output is checked for each breast. If the milk output is good, move to step 142. If the milk output is not good, move to step 144. At step 144, the user adjusts the pumping parameters (vacuum strength 98, 100 and/or cycles per minute 94, 96) to maximize output for the lagging breast. Then move to step 142. Again, the system 110 monitors and records data and information to ultimately determine the optimal settings for the particular user.

At step 142, the user refines the settings as needed to maximize milk output per side. At step 146, the user finishes pumping, and at step 148, the system 110 retains the maximum settings for the next session. Those retained settings may include dynamic settings throughout the pumping session. The recorded data and information is stored in the database 114 for further determination.

The flow chart disclosed herein exemplify the type of controls and to some extent the software program that can be utilized to control the breast pump 60 and/or the breast pump system 110, separately or when integrated. These flow charts represent the software program, and can be used in combination with each other or separately, to effect the functionality of the disclosed improved breast pump 60 and breast pump system 110.

It will be understood that the embodiments of the present disclosure, which have been described, are illustrative of some of the applications of the principles of the present disclosure. Although numerous embodiments of this disclosure have been described above with a certain degree of particularity, those skilled in the art could alter the disclosed embodiments without departing from the spirit or scope of this disclosure.

All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosed system and methods.

Additionally, joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosed apparatus, system and methods as disclosed herein.

1. An improved double pump breast pump comprising:
A housing, said housing configured to secure a first pump and a second pump;
a user interface, said user interface comprising a plurality of switches and a display;
said plurality of switches coupled to said first pump and to said second pump to control and adjust said first pump and said second pump independently from each other;
said display configured to show a first plurality of parameters of said first pump and a second plurality of parameters of said second pump during a breast pumping session;
a first breast flange, said first breast flange configured to be secured over a first nipple of a first breast;
a second breast flange, said second breast flange configured to be secured over a second nipple of a second breast;
a first tubing, said first tubing configured to be connected between said pump and said first flange;
a second tubing, said second tubing configured to be connected between said pump and said second flange;
such that when said first pump is coupled to said first flange with said first tubing, and said second pump is coupled to said second flange with said second tubing, the user interface can separately control the pumping of said first breast based on the first plurality of parameters of said first pump, and control the pumping of said second breast based a second plurality of parameters of said second pump, during a breast pumping session.

2. The improved double pump breast pump of claim 1, wherein said first plurality of parameters of said first pump comprises a first vacuum for said first pump.

3. The improved double pump breast pump of claim 1, wherein said second plurality of parameters of said second pump comprises a second vacuum for said second pump.

4. The improved double pump breast pump of claim 1, wherein said first plurality of parameters of said first pump comprises a first cycle for said first pump.

5. The improved double pump breast pump of claim 1, wherein said second plurality of parameters of said first pump comprises a second cycle for said second pump.

6. An improved double pump breast pump comprising:
A housing, said housing configured to secure a printed circuit board, a first pump and a second pump, said printed circuit board configured to secure a microprocessor, said microprocessor configured to control said first pump and said second pump;
a user interface, said user interface comprising switches; said switches coupled to said microprocessor to control and adjust one or more parameters separately for said first pump and said second pump;
a display, said display configured to show a plurality of conditions of said first pump and said second pump separately during a breast pumping session;
a first breast flange, said first breast flange configured to be secured over a first nipple of a first breast;
a second breast flange, said second breast flange configured to be secured over a second nipple of a second breast;
a first tubing, said first tubing configured to be connected between said pump and said first flange;
a second tubing, said second tubing configured to be connected between said pump and said second flange;
such that when said first pump is coupled to said first flange with said first tubing, and said second pump is coupled to said second flange with said second tubing, the user interface can separately control the one or more parameters for pumping said first breast through said first nipple using the first pump, and said second breast through said second nipple using the second pump.

7. The improved double pump breast pump of claim 6, wherein said one or more parameters comprise a first vacuum for said first pump.

8. The improved double pump breast pump of claim 6, wherein said one or more parameters comprise a second vacuum for said second pump.

9. The improved double pump breast pump of claim 6, wherein said one or more parameters comprise a first cycle for said first pump.

10. The improved double pump breast pump of claim 6, wherein said one or more parameters comprise a second cycle for said second pump.
